(19) United States
(12) Patent Application Publication
(10) Pub. No.: US 2006/0128605 A1
(43) Pub. Date: Jun. 15, 2006

(54) Preventives/remedies for snore or respiratory disturbances during sleep

(75) Inventors: Susumu Shibahara, Kawasaki-shi (JP); Ryosei Sakai, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 11/280,354

(22) Filed: Nov. 17, 2005

Related U.S. Application Data
(63) Continuation of application No. PCT/JP04/07106, filed on May 19, 2004.

(30) Foreign Application Priority Data
May 19, 2003 (JP) 141124/2003

(51) Int. Cl. A61K 38/16; A61K 31/198

(52) U.S. Cl. 514/2; 514/561

(57) ABSTRACT

Compounds which have an agonist action on a glycine receptor and compounds capable of being present as glycine in living organisms are effective for the prophylaxis or treatment of snoring or respiratory disorders during sleep. Foods and diets which contain such a compound are also effective for the prophylaxis or palliation of snoring or respiratory disorders during sleep.

### CROSS REFERENCES TO RELATED APPLICATIONS

This application claims priority to International Patent Application No. PCT/JP2004/007106, filed on May 19, 2004, and Japanese Patent Application No. 2003-141124, filed on May 19, 2003, both of which are incorporated herein by reference in their entireties.

### BACKGROUND OF THE INVENTION

### Field of the Invention

The present invention relates to agents for the prophylaxis or treatment of snoring or a respiratory disorder during sleep. The present invention also relates to foods and diets effective for the prophylaxis or palliation of snoring or a respiratory disorder during sleep.

### DISCUSSION OF THE BACKGROUND

Snoring occurs when the airway is constricted or obstructed due to the decreased tension of the muscle of throat or tongue during sleep and the airway mucous membrane of the throat and the like vibrates. The causes of snoring include obesity, drinking, intake of a hypnotic, aging, nasal diseases such as adenoid and the like, disorders in the throat, and the like. Snoring not only disturbs one's housemate's sleep, thus causing trouble for others, but also prevents deep sleep and causes daytime sleepiness, a decline in concentration, activity and memory, and mental instability. Therefore, prevention of snoring is important for ensuring the good sleep of a housemate as well as the snorer, enhancing vitality during daytime, and maintaining good health.

Approximately 10-20% of snorers show an episode of repeated, intermittent sleep apnea (apnea during sleep) and such episodes are clinically referred to as sleep apnea syndrome. Two million latent patients are estimated to be present in Japan alone.

As the etiology of sleep apnea, disorders in the respiratory central nervous system can be mentioned, besides the afore-mentioned airway obstruction.

It is known that snoring and sleep apnea not only prevent daytime activities but also cause traffic accidents due to daytime somnolence, and cause various disorders resulting from the effects of oxygen shortage in the body, which is caused by temporarily interrupted breathing, on the circulatory and central nervous systems. For example, the association of snoring with hypertension, cerebral infarction, ischemic heart disease, arrhythmia and sudden death has been pointed out, and they are considered to be diseases that require prevention and treatment.

When snoring and sleep apnea are considered to be associated with obesity, drinking, continued use of a hypnotic-analgesic-tranquilizer and the like, a reform of the subject's lifestyle such as weight loss, nondrinking, withdrawal from hypnotic-analgesic-tranquilizer and the like becomes the primary treatment and prevention. For a more positive treatment, the use of intraoral accessory and a surgical treatment by operation may be employed, though apnea may not be improved in some cases. For a more certain cure, a therapy by feeding air through a nose mask to always maintain positive pressure in the upper airway or CPAP (Continuous Positive Airway Pressure) has been recently developed, though compliance is not necessarily good.

As a treatment method using a pharmaceutical agent, drugs such as steroids, etc. may be effective as far as snoring and sleep apnea which are caused by inflammation of the airway or allergy. In addition, a hypotensive diuretic (acetazolamide) may be used for the treatment, but the treatment is not necessarily effective.

Moreover, there is a report indicating that pharmaceutical agents that suppress activities of glutamic acid neuron in the central nervous system, namely, inhibitors of glutamic acid receptor or glycine receptor, and antagonists of glutamic acid or glycine are potentially effective for the treatment of sleep apnea syndrome (see, WO00/51590), and the effectiveness of riluzole, which is a glutamic acid antagonist, has been confirmed through animal experiments using rats. However, antagonists of glutamic acid or glycine having such efficacy have not been put to practical use in clinical situations and the effectiveness thereof in human has not been confirmed.

Therefore, there remains a need for effective therapeutic drugs, food and the like for snoring and/or sleep apnea.

### SUMMARY OF THE INVENTION

Accordingly, it is one object of the present invention to provide novel pharmaceutical agents for the prophylaxis or treatment of respiratory disorders during sleep.

It is another object of the present invention to provide novel pharmaceutical agents for preventing or reducing snoring.

It is another object of the present invention to provide novel foods and/or diets for the prophylaxis or palliation of snoring and/or a respiratory disorder during sleep.

It is another object of the present invention to provide novel methods for preventing or reducing snoring.

It is another object of the present invention to provide novel foods and/or diets for preventing or reducing snoring.

These and other objects, which will become apparent during the following detailed description, have been achieved by the inventor's discovery that the administration of glycine suppresses the occurrence of snoring and further suppresses sleep apnea and reduced arterial blood oxygen saturation (SpO₂) due to sleep apnea. This effect can be also achieved by serine which increases, upon metabolism in the body via glycine, the amount of glycine in the body, and peptides and proteins that contain glycine and/or serine as component amino acid(s) in a high ratio. In other words, any compound can express this effect as long as it can be present as glycine in living organisms. This effect can be also achieved by a compound having an agonist action on the glycine receptors (hereinafter to be also referred to as “glycine receptor agonist” ).

The present invention has been completed based on such findings and the confirmation that a compound that can be present as glycine in living organisms (hereinafter to be referred to as “glycine etc.”) and glycine receptor agonists can be used for the prophylaxis or treatment of snoring or a respiratory disorder during sleep, and that glycine etc. and glycine receptor agonists can be used for the production of an agent for the prophylaxis or treatment of snoring or a respiratory disorder during sleep.

The glycine etc. and glycine receptor agonists can be added to a food or diet for utilization for the prophylaxis or palliation of snoring or a respiratory disorder during sleep.

Accordingly, the present invention provides the following:

(1) An agent for the prophylaxis or treatment of snoring or a respiratory disorder during sleep, which comprises a compound having an agonist action on a glycine receptor as an active ingredient.

(2) An agent for the prophylaxis or treatment of snoring or a respiratory disorder during sleep, which comprises a compound capable of being present as glycine in living organisms as an active ingredient.

(3) The agent of the above-mentioned (2), wherein the compound capable of being present as glycine in living organisms is selected from glycine, serine, a peptide containing glycine and/or serine as component amino acid(s), and a protein containing glycine and/or serine as component amino acid(s).

(4) The agent of any of the above-mentioned (1)-(3), wherein the respiratory disorder during sleep is hypopnea or apnea.

(5) The agent of the above-mentioned (4), wherein the hypopnea or apnea during sleep is obstructive hypopnea or obstructive sleep apnea, or central nervous system hypopnea or central nervous system apnea.

(6) The agent of any of the above-mentioned (1)-(3), which is an agent for the prophylaxis or treatment of sleep apnea syndrome.

(7) The agent of any of the above-mentioned (1)-(3), wherein the respiratory disorder during sleep is a respiratory disorder caused by asthma.

(8) The agent of the above-mentioned (2) or (3), which is used for human or animal, wherein the intake or dose is 0.06 to 2500 mg/kg/day based on the free form of glycine, or 0.1 to 4000 mg/kg/day based on the free form of serine.

(9) A food for the prophylaxis or palliation of snoring or a respiratory disorder during sleep, which comprises a compound having an agonist action on a glycine receptor.

(10) A food for the prophylaxis or palliation of snoring or a respiratory disorder during sleep, which comprises a compound capable of being present as glycine in living organisms.

(11) The food of the above-mentioned (9) or (10), which is in the form of a beverage.

(12) The food of any of the above-mentioned (9)-(11), which is a supplement.

(13) The food of any of the above-mentioned (9)-(11), which is a food with health claims.

(14) A diet for the prophylaxis or palliation of snoring or a respiratory disorder during sleep, which comprises a compound having an agonist action on a glycine receptor.

(15) A diet for the prophylaxis or palliation of snoring or a respiratory disorder during sleep, which comprises a compound capable of being present as glycine in living organisms.

(16) A package comprising the agent of any of the above-mentioned (1)-(8) together with a written matter describing items relating to the use of the agent for the prophylaxis or treatment of snoring or a respiratory disorder during sleep.

(17) A method for the prophylaxis or treatment of snoring or a respiratory disorder during sleep, which comprises administering a compound having an agonist action on a glycine receptor to a human or animal in need of said prophylaxis or treatment.

(18) A method for the prophylaxis or treatment of snoring or a respiratory disorder during sleep, which comprises administering a compound capable of being present as glycine in living organisms to a human or animal in need of said prophylaxis or treatment.

(19) The method of the above-mentioned (18), wherein the compound capable of being present as glycine in living organisms is selected from glycine, serine, a peptide containing glycine and/or serine as component amino acid(s), and a protein containing glycine and/or serine as component amino acid(s).

(20) The method of any of the above-mentioned (17)-(19), wherein the respiratory disorder during sleep is hypopnea or apnea.

(21) The method of the above-mentioned (20), wherein the hypopnea or apnea during sleep is obstructive hypopnea or obstructive apnea, or central nervous system hypopnea or central nervous system apnea.

(22) The method of any of the above-mentioned (17)-(19), which is a method for the prophylaxis or treatment of sleep apnea syndrome.

(23) The method of any of the above-mentioned (17)-(19), wherein the respiratory disorder during sleep is a respiratory disorder caused by asthma.

(24) The method of the above-mentioned (18) or (19), which comprises administering, to a human or animal, the compound capable of being present as glycine in living organisms in an amount of 0.06 to 2500 mg/kg/day based on the free form of glycine or 0.1 to 4000 mg/kg/day based on the free form of serine.

(25) A method for the prophylaxis or palliation of snoring or a respiratory disorder during sleep, which comprises feeding a food containing a compound having an agonist action on a glycine receptor to a human or animal in need of said prophylaxis or palliation.

(26) A method for the prophylaxis or palliation of snoring or a respiratory disorder during sleep, which comprises feeding a food containing a compound capable of being present as glycine in living organisms to a human or animal in need of said prophylaxis or palliation.

(27) The method of the above-mentioned (25) or (26), wherein the food is in the form of a beverage.

(28) The method of any of the above-mentioned (25)-(27), wherein the food is a supplement.

(29) The method of any of the above-mentioned (25)-(27), wherein the food is a food with health claims.

(30) A method for the prophylaxis or palliation of snoring or a respiratory disorder during sleep, which comprises feeding a diet containing a compound having an agonist action on a glycine receptor to an animal in need of said prophylaxis or palliation.

(31) A method for the prophylaxis or palliation of snoring or a respiratory disorder during sleep, which comprises feeding a diet containing a compound capable of being present as glycine in living organisms to an animal in need of said prophylaxis or palliation.

(32) Use of a compound having an agonist action on a glycine receptor for the production of an agent for the prophylaxis or treatment of snoring or a respiratory disorder during sleep.

(33) Use of a compound capable of being present as glycine in living organisms for the production of an agent for the prophylaxis or treatment of snoring or a respiratory disorder during sleep.

(34) The use of the above-mentioned (33), wherein the compound capable of being present as glycine in living organisms is selected from glycine, serine, a peptide containing glycine and/or serine as component amino acid(s), and a protein containing glycine and/or serine as component amino acid(s).

(35) The use of any of the above-mentioned (32)-(34), wherein the respiratory disorder during sleep is hypopnea or apnea.

(36) The use of the above-mentioned (35), wherein the hypopnea or apnea during sleep is obstructive hypopnea or obstructive apnea, or central nervous system hypopnea or central nervous system apnea.

(37) The use of any of the above-mentioned (32)-(34) for the production of an agent for the prophylaxis or treatment of sleep apnea syndrome.

(38) The use of any of the above-mentioned (32)-(34), wherein the respiratory disorder during sleep is a respiratory disorder caused by asthma.

(39) The use of the above-mentioned (33) or (34) for the production of an agent for a human or animal for the prophylaxis or treatment of snoring or a respiratory disorder during sleep, wherein the intake or dose is 0.06 to 2500 mg/kg/day based on the free form of glycine, or 0.1 to 4000 mg/kg/day based on the free form of serine.

(40) Use of a compound having an agonist action on a glycine receptor for the production of a food for the prophylaxis or palliation of snoring or a respiratory disorder during sleep.

(41) Use of a compound capable of being present as glycine in living organisms for the production of a food for the prophylaxis or palliation of snoring or a respiratory disorder during sleep.

(42) The use of the above-mentioned (40) or (41), wherein the food is in the form of a beverage.

(43) The use of any of the above-mentioned (40)-(42), wherein the food is a supplement.

(44) The use of any of the above-mentioned (40)-(42), wherein the food is a food with health claims.

(45) Use of a compound having an agonist action on a glycine receptor for the production of a diet for the prophylaxis or palliation of snoring or a respiratory disorder during sleep.

(46) Use of a compound capable of being present as glycine in living organisms for the production of a diet for the prophylaxis or palliation of snoring or a respiratory disorder during sleep.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows a mouth-nose breathing pattern (upper line) and simultaneously measured profile (lower line) of arterial blood oxygen saturation (SpO₂, % saturation) for 6 minutes when typical apnea was observed. A temperature increase in a temperature sensor, set on a mouth-nose part, due to expiration and a temperature decrease by inspiration is shown as a mouth-nose breathing pattern. The shadow shows apnea (upper line) and SpO₂ decrease (lower line). An SpO₂ decrease is observed after occurrence of apnea;

FIG. 2 shows a profile (lower line) of arterial blood oxygen saturation (SpO₂, % saturation) from falling asleep to waking up when a glycine tablet was taken and an SpO₂ profile (upper line) of control free of administration, wherein a bar shows 80 minutes;

FIG. 3 shows a typical example (lower line) of tracheal sound (snoring noise) when a glycine tablet was taken and a tracheal sound (upper line) of a control free of administration, wherein amplitude shows the level of tracheal sound and a bar shows 5 minutes;

FIG. 4 is a graph showing the results of measurement of AHI (apnea hypopnea index) during sleep after taking glycine and after taking placebo in Example 2. The left graph of FIG. 4 is an average value of AHI after taking glycine (black column graph) and an average value of AHI after taking placebo (outline column graph) of the entire period (Total) of the rem sleep stage and the nonrem sleep stage, the rem sleep stage (REM) and the nonrem sleep stage (NREM) in the entire sleep stage. The right graph of FIG. 4 shows an average value of AHI after taking glycine (black column graph) and an average value of AHI after taking placebo (outline column graph) of Total, REM and NREM in the early stage of sleep (3 hours); and

FIG. 5 is a graph showing the results of measurement of AHI during sleep after taking glycine and after taking placebo in Example 2. The left graph of FIG. 5 shows an average value of AHI after taking glycine (black column graph) and an average value of AHI after taking placebo (outline column graph) of test recipients of 40 years old or over and the right graph of FIG. 5 graph shows an average value of AHI after taking glycine (black column graph) and an average value of AHI after taking placebo (outline column graph) of test recipients of 40 years old or over, who show an obesity index (BMI) exceeding 28, wherein * shows significance.

### DETAILED DESCRIPTION OF THE PREFERRED EMBODIMENTS

The mode of embodiment of the present invention is explained below.

As the snoring and respiratory disorders during sleep that are the targets of the agents for the prophylaxis or treatment of snoring or a respiratory disorder during sleep of the present invention (hereinafter to be simply referred to as “the agents for the prophylaxis or treatment of the present invention”), snoring, obstructive or central nervous system hypopnea as seen in what is called a sleep apnea syndrome, apnea, as well as hypopnea and apnea caused by other etiology disease such as asthma and the like can be mentioned.

The glycine receptor agonist is a substance that binds to a glycine receptor and shows an effect the same as or similar to the effect of glycine.

As the glycine receptor agonist, for example, aminomethanesulfonic acid, D-alanine, L-alanine, β-alanine, taurine, hypotaurine, dodecylbenzenesulfonate, penicillin G, Chlormethiazole, Ivermectin, and the like can be mentioned.

The compound capable of being present as glycine in living organisms is, not to mention glycine, a concept encompassing compounds like proteins that produce glycine by cleaving a peptide bond after administration, and compounds that produce glycine in living organisms after administration, such as serine that produces glycine by metabolism (can be a compound that produces glycine during metabolism) and the like.

As the compound capable of being present as glycine in living organisms, for example, glycine and/or serine, peptide which contain glycine and/or serine as component amino acid(s) at a high ratio, proteins which contain glycine and/or serine as component amino acid(s) at a high ratio, and the like can be mentioned.

Serine is metabolized in the body via glycine to increase the amount of glycine in the body.

The peptide containing glycine and/or serine as component amino acid(s) and the protein containing glycine and/or serine as component amino acid(s) are decomposed into glycine and/or serine through digestion etc. to increase the amount of glycine in the body.

The peptide containing glycine and/or serine as component amino acid(s) preferably contains higher amounts of glycine and/or serine as component amino acid(s) and, for example, a peptide having a content of glycine and serine of 0.1 to 1 g/g peptide, preferably 0.3 to 1 g/g peptide, as the total amount of glycine and serine can be mentioned.

The protein containing glycine and/or serine as component amino acid(s) preferably contains higher amounts of glycine and/or serine as component amino acid(s) and, for example, a protein having a content of glycine and serine of 0.1 to 1 g/g protein, preferably 0.3 to 1 g/g protein, as the total amount of glycine and serine can be mentioned.

As the peptide or protein containing glycine and/or serine as component amino acid(s), for example, silk, collagen, gelatin, peptides obtained by chemical synthesis, and the like can be mentioned.

The glycine etc. and glycine receptor agonists may be in the form of salts, and the terms “glycine,” “serine,” “a peptide containing glycine and/or serine as component amino acid(s),” “a protein containing glycine and/or serine as component amino acid(s),” and “a compound having an agonist action on a glycine receptor (glycine receptor agonist)” in the present description are concepts also encompassing salts.

Such salts are not particularly limited as long as they are pharmacologically acceptable and, for example, salts with an inorganic acid or an organic acid can be mentioned. As the salts with an inorganic acid, for example, salts with hydrochloric acid, hydrobromic acid, nitric acid, sulfuric acid, phosphoric acid, etc. can be mentioned. As the salts with an organic acid, for example, salts with formic acid, acetic acid, trifluoroacetic acid, propionic acid, lactic acid, tartaric acid, oxalic acid, fumaric acid, maleic acid, citric acid, malonic acid, methanesulfonic acid, etc. can be mentioned. In addition, salts with a base can be also used. As the salts with base, for example, alkali metal salts such as sodium salt, potassium salt, etc.; alkaline earth metal salts such as calcium salt, magnesium salt, etc.; and the like can be mentioned.

The agent for the prophylaxis or treatment of the present invention is administered in the form of a known pharmaceutical preparation or a pharmaceutical preparation to be developed in the future, such as a form for oral administration or parenteral administration, and oral administration is convenient and preferable. As the oral agent, solid agents such as powders, granules, tablets, (micro)capsules, etc.; solutions such as syrups, juices, etc.; emulsions; suspensions; and the like can be mentioned. As the parenteral agent, injection, transbowel, transdermal and inhalation preparations can be mentioned. These preparations can be processed into instantaneous or slow acting (sustained-release) preparations.

For production of these preparations, a method known per se or a method to be developed in the future can be appropriately employed. For the production of the preparation, an appropriate pharmaceutically acceptable carrier such as a substance used for general preparations can be used as auxiliary agent according to the dosage form of each preparation. As the substance used for general preparations, for example, excipients, diluents, additives, disintegrants, binders, coating agents, lubricants, gliding agents, glossing agents, flavors, sweetening agents, solubilizers, and the like can be mentioned. More concrete examples of the substance used for general preparations include magnesium carbonate, titanium dioxide, lactose, mannitol, dextrin and other sugars, talc, milk protein, gelatin, starch, cellulose (crystalline cellulose) and derivatives thereof, animal and vegetable oils, polyethylene glycol, glyceride, fine silicon dioxide, solvents such as sterilized water, monovalent alcohols (e.g., ethanol etc.) and polyhydric alcohols (e.g., glycerol etc.), vanilla flavor, and the like.

The content of the active ingredient (glycine etc., glycine receptor agonist) in the agent for the prophylaxis or treatment of the present invention in the above-mentioned dosage form is not particularly limited and can be appropriately adjusted to contain an amount necessary for providing the efficacy.

The agent for the prophylaxis or treatment of the present invention can be formed as a commercial package containing a written matter which describes items relating to the use of the agent for the prophylaxis or treatment.

The recipients of the intake or administration are humans and animals (including experimental animals). The intake or dose of glycine etc. is about 0.06 to 2500 mg/kg/day, more preferably about 0.25 to 625 mg/kg/day, and still more preferably about 1.25 to 125 mg/kg/day, based on the free form of glycine. Alternatively, it is about 0.1 to 4000 mg/kg/day, more preferably about 0.4 to 2400 mg/kg/day, and still more preferably about 1.6 to 600 mg/kg/day, based on the free form of serine.

While the intake or dose of the glycine receptor agonist varies depending on the kind of pharmaceutical agent, it is preferably 0.001 to 600 mg/kg/day.

In the present invention, the daily dose is administered orally or parenterally at once or in 2 to 4 portions at appropriate intervals. For one time administration, it is preferably taken before going to bed.

According to the agent for the prophylaxis or treatment of the present invention, snoring and sleep disorders caused by hypopnea and apnea during sleep can be prevented and the distress due to sleeplessness can be reduced.

According to the agent for the prophylaxis or treatment of the present invention, moreover, snoring or sleep disorders due to hypopnea or apnea during sleep can be prevented, daytime somnolence, scattering of attention, decline of memory, reduced efficiency of work, accidents, frustration, short temper, morning headaches, dysthymia, hyposexuality, hallucination, deafness, and the like due to sleep disorder, as well as abnormal behavior during night due to sleep disorder can be prevented, and further, social maladjustment due to various symptoms caused by these sleep disorders and anxiety or melancholia can be prevented.

According to the agent for the prophylaxis or treatment of the present invention, moreover, the onset of cerebrovascular disorder, hypertension, polycythemia, pulmonary hypertension, pulmonary heart disease, congestive heart failure, ischemic heart disease, arrhythmia, respiratory failure, sudden death, diabetes, obesity, cognitive impairment, mental disorder, and dementia, which are complications of sleep apnea, can be prevented.

The agent for the prophylaxis or treatment of the present invention is effective for improving snoring and respiratory disorders (particularly hypopnea or apnea) during the entire sleep hours from initiation of sleep to wakefulness, and is characterized by the particularly high effectiveness in the early stage of sleep. In addition, the agent for the prophylaxis or treatment of the present invention is effective in remarkably improving snoring or respiratory disorders (particularly hypopnea or apnea) during sleep in individuals of 40 years old or above, particularly individuals of 40 years old or above and with obesity (e.g., obesity of over 28 in BMI).

The “food” of the present invention means foods in general (including beverage), general foods including what is called health food, foods for specified health uses and foods with nutrient function claims, as defined in the food with health claims system by the Ministry of Health, Labour and Welfare, and further, supplements.

In the present invention, the glycine etc. and glycine receptor agonist can be utilized by addition to various processed foods, general foods (e.g., beverage), foods with health claims (e.g., food for specified health uses, food with nutrient function claims) or diets, or as supplements, for the purpose of prophylaxis or palliation of snoring or a respiratory disorder during sleep.

The food of the present invention can be produced by a method known per se using a carrier acceptable as appropriate food, such as various foodstuffs for processing, seasonings, flavors, sweeteners, and the like as powders, tablets, capsules, drinks, and the like. The carrier acceptable as food includes the aforementioned pharmaceutically acceptable carriers.

For production of the food of the present invention, other additives for nutritional support such as vitamins and the like can be used.

The diet of the present invention can be produced by a method known per se using a carrier acceptable as appropriate diet, such as general foodstuffs and the like. The carrier acceptable as diet includes the aforementioned pharmaceutically acceptable carriers. For administration to animals, the aforementioned method for administration to human is employed, or administration upon addition to general diet is employed.

While the amount of glycine etc., glycine receptor agonist to be contained in the food of the present invention is not particularly limited, it is preferably set to such an amount that makes the daily intake fall within the range of the above-mentioned dose of the agent for the prophylaxis or treatment of the present invention.

Other features of the invention will become apparent in the course of the following descriptions of exemplary embodiments which are given for illustration of the invention and are not intended to be limiting thereof.

### EXAMPLES

### Example 1

A test recipient took a glycine tablet and a tablet containing L-cystine as an amino acid other than glycine (hereinafter to be abbreviated as a control tablet) before bedtime and the family member evaluated snoring of the test recipient during sleep. In addition, sleep apnea and arterial blood oxygen saturation (SpO₂) were evaluated using a sleep tester LT-200 manufactured by FUKUDA DENSHI Co., Ltd. (Japan). The test recipient was a 38-year-old healthy male (body weight 82 kg, height 170 cm, body mass index (BMI) 28.3), and frequent occurrence of apnea during sleep was observed by the family member. The test recipient was completely free of regular medication, and had no habit of taking nutritional supplements or nutritional supports. During the test period, no pharmaceutical products, no nutritional supplement and no nutritional support, other than the glycine tablet and the control tablet, were taken. The test recipient took an appropriate number (2-4) of the glycine tablets or the control tablets, and interviewed a family member the next morning as to the presence or absence of snoring and the level thereof, without informing the family member of the kind of the tablets and whether he took the tablets.

Preparation of Glycine Tablet:

Glycine (3,675 g), dextrin (1,725 g), and glyceride (375 g) were mixed, crystalline cellulose (1,500 g) and 70% ethanol at a weight ratio of 22% were added, and the mixture was kneaded and extrusion-granulated. The obtained granulation product was dried to a water content of 2.28% and sized to give 16 mesh PASS particles. Then, fme silicon dioxide (150 g) and vanilla flavor (75 g) were added and mixed. The obtained mixture was tableted under the conditions of 11 mmφ, 500 mg, tableting pressure 2.2 ton, 20 rpm, average hardness 10 kg. By the above operation, 11 mm diameter tablets containing 245 mg of glycine per tablet were produced.

Preparation of Control Tablet:

L-cystine (2,625 g), L-teanin (1,050 g), dextrin (1,710 g), and aspartam (15 g) were mixed, crystalline cellulose (1,500 g) and 70% ethanol at a weight ratio of 30% were added, and the mixture was kneaded and extrusion-granulated. The obtained granulation product was dried to a water content of not more than 1.6% and sized to give 16 mesh PASS particles. Then, fine silicon dioxide (37.5 g), glyceride (487.5 g) and vanilla flavor (75 g) were added and mixed. The obtained mixture was tableted under the conditions of 11 mmφ, 500 mg, tableting pressure 2.0 ton, 20 rpm, average hardness 10 kg. By the above operation, 11 mmφ diameter control tablets (cysteine-teanin tablets) containing 175 mg of L-cystine and 70 mg of L-teanin per tablet were produced.

Evaluation of Snoring:

The presence or absence of snoring during sleep was evaluated the next morning by a family member into three levels of minus (−; snoring was not heard or like sleep breath), plus (+; medium level snoring), two plus (++; extremely noisy and severe snoring). The presence or absence of snoring when the glycine tablets and control tablets were taken before bedtime, and nothing was taken before bedtime was counted. For comparison between the respective treatments in terms of the results of the snoring evaluation and drinking frequency, the X square test was used and a critical value of p<0.05 was taken as statistical significance.

Evaluation of Apnea:

The arterial blood oxygen saturation (SpO₂) and air flow in the mouth-nose during sleep were measured using a sleep tester LT-200 manufactured by FUKUDA DENSHI Co., Ltd. The arterial blood oxygen saturation (SpO₂) was measured using a sensor attached to the tip of the second finger of the left hand, and a decrease in the arterial blood oxygen saturation (SpO₂) to not more than 93% which lasted for not less than 10 seconds was taken as SpO₂ decrease. The number of SpO₂ decrease per one day and per one hour and the minimum SpO₂ of one day were determined. The air flow in the mouth-nose part was measured by a thermocapsule method, and a decrease in the flow in the mouth-nose part to not more than 50% which lasted for not less than 10 sec was defined as hypopnea. The occurrence of SpO₂ decrease along with hypopnea was counted as apnea and the number of apnea per one day and per one hour was determined. Simultaneously, tracheal sounds during sleep were picked up through a mike set near laryngeal prominence.

Experiment 1.

For evaluation of whether or not snoring is reduced by taking a glycine tablet, the presence or absence of snoring during sleep was observed for 27 days. After an observation period (3 days) free of administration, the presence or absence of snoring was observed after administration of 2 glycine tablets (containing glycine 490 mg) for 9 days, and 4 glycine tablets (containing glycine 980 mg) for 8 days before bedtime, and also observed for 7 days without administration of glycine tablet (see, Table 1).

Results of Experiment 1.

When the glycine tablet was not administered, extremely annoying snoring (an evaluation of two plus, ++) was observed for 5 days out of 10 days (frequency 50%) (see, Table 1). The number of days when an evaluation two plus snoring was observed reduced to 2 days out of 9 days (frequency 22%) when glycine tablets (2 tablets) were administered before bedtime, and was not observed when glycine tablets (4 tablets) were administered for 8 days. This decrease was significant as compared to non-administration of glycine tablet (p<0.05). On the other hand, the number of days when snoring was absent, or at the sleep breath level (an evaluation of minus, −) was 3 days out of 10 days (frequency 30%) when the glycine tablet was not administered, but dose-dependently increased to 4 days out of 9 days (frequency 44%) by glycine tablet (2 tablets) administration, and 6 days out of 8 days (frequency 75%) by administration of 4 tablets. Since drinking of alcoholic beverages is known to encourage the occurrence of snoring, the number of days of drinking is shown for glycine non-administration, 2 tablet administration and 4 tablet administration (see, Table 1). Since drinking was highly frequent at the time of glycine 2 tablet and 4 tablet administrations, the reason for suppression of snoring is not reduced drinking, but, instead, administration of the glycine tablet clearly suppressed the occurrence of snoring.

TABLE 1

| glycine | | | | | | number of |
|---|---|---|---|---|---|---|
| (number of | total days of | | snoring evaluation results | | | days of |
| tablets taken) | observation | | − | + | ++ | drinking |
| 0 | 10 | days | 3 days | 2 days | 5 days | 6 days |
| | | | (30%) | (20%) | (50%) | (60%) |
| 2 | 9 | days | 4 days | 3 days | 2 days | 9 days |
| | | | (44%) | (33%) | (22%) | (100% a)) |
| 4 | 8 | days | 6 days | 2 days | 0 day | 7 days |
| | | | (75%) | (25%) | (0% a)) | (88%) |

Glycine tablets (O to 4 tablets) were administered before bedtime, and the presence or absence of snoring was evaluated next morning by a family member. For evaluation results of snoring, the number of days when snoring was not observed (evaluation minus (−)), and the number of days when moderate level of snoring (evaluation plus (+)), and severe level of snoring (evaluation two plus (++)) were observed are shown. In the parentheses, percentages of the number of days when the evaluation was −, +, or ++ of the total observation days are shown. Simultaneously, the number of drinking days (the percentage of the total number of days of observation in parentheses) is shown. a): Significant (p<0.05) relative to the absence of glycine tablet administration.
Experiment 2.

The question of whether or not the control tablet shows a similar snoring suppressive effect as the glycine tablet was then examined. After an observation period free of administration (5 days) of any tablet, the presence or absence of snoring was observed for 10 days after the control tablets (4 tablets) were administered before bedtime, and for 5 days without administration of the tablet. Thereafter, the presence or absence of snoring was observed for 9 days after the glycine tablets (4 tablets) (containing glycine 980 mg) were administered before bedtime.

Results of Experiment 2.

As shown in Table 2, severe snoring (evaluation two plus, ++) was observed for 4 days (frequency 40%) out of 10 days of control tablet administration, and the frequency was not different from the absence of administration of the tablet. In contrast, severe snoring was not observed for the 9 days of glycine tablet administration. Conversely, the number of days when the snoring evaluation result was minus was 3 days (frequency 30%) and 2 days (frequency 20%) out of the 10 days each of the absence of administration and control tablet administration, respectively. When the glycine tablet was administered, it increased to 6 days (frequency 67%) out of 9 days. From the above results, it is clear that the glycine administration suppressed snoring.

TABLE 2

| number of | | | | | | number of |
|---|---|---|---|---|---|---|
| tablets | total days of | | snoring evaluation results | | | days of |
| taken | observation | | − | + | ++ | drinking |
| no admin- | 10 | days | 3 days | 4 days | 3 days | 4 days |
| istration | | | (30%) | (40%) | (30%) | (40%) |
| glycine tablet | 9 | days | 6 days | 3 days | 0 days | 7 days |
| | | | (67% a)) | (33%) | (0% a)) | (78%) |
| control tablet | 10 | days | 2 days | 4 days | 4 days | 7 days |
| | | | (20%) | (40%) | (40%) | (70%) |

With regard to each case of glycine tablet administration, control tablet administration, and non-administration before bedtime, the presence or absence of snoring was evaluated the next morning by a family member. For the snoring evaluation results, each number of days when snoring was not observed (evaluation minus (−)), moderate level of snoring (evaluation plus (+)), and severe level of snoring (evaluation two plus (++)) was observed is shown. In the parentheses, percentages of the number of days when the evaluation was −, +, ++ of the total observation days are shown. Simultaneously, the number of drinking days (percentages of the total number of days of observation in parentheses) is shown. a): Significant (p<0.05) relative to the control tablet administration.

When the drinking frequency in Experiment 1 and Experiment 2 was compared between glycine tablet administration and non-administration, the frequency was higher when associated with administration. Since drinking is generally considered to potentiate snoring, high drinking frequency associated with glycine tablet administration is assumed not to affect the conclusion that “glycine suppresses the occurrence of snoring.” To further confirm this, the relationship between the presence or absence of drinking and snoring evaluation results was analyzed (see, Table 3) with regard to all cases of Experiment 1 and Experiment 2 when glycine tablet was not administered. The number of days when severe snoring (evaluation two plus, ++) was observed was 3 days (frequency 23%) out of 13 days without drinking, but reached 9 days (frequency 53%) out of 17 days of drinking. Conversely, the number of days when snoring was not heard or it was of a sleep breath level (evaluation minus, −) was 6 days (frequency 46%) out of 13 days without drinking, but significantly decreased to 2 days (frequency 12%) out of 17 days with drinking. From the foregoing, the conventional finding that drinking potentiates snoring was re-confirmed. Therefore, it is clear that the present test results relating to the glycine tablet administration effect were not brought about by the difference in the drinking frequency but because “glycine administration suppressed snoring.”

TABLE 3

| | total days of | snoring evaluation results | | |
|---|---|---|---|---|
| drinking | observation | − | + | ++ |
| no | 13 days | 6 days | 4 days | 3 days |
| | | (46%) | (31%) | (23%) |
| yes | 17 days | 2 days | 6 days | 9 days |
| | | (12% a)) | (35%) | (53%) |

The snoring evaluation results of the presence and absence of drinking are shown in Table 3 above. For the snoring evaluation results, each number of days when snoring was not observed (evaluation minus (−)), moderate level of snoring (evaluation plus (+)), and severe level of snoring (evaluation two plus (++)) was observed is shown. In the parentheses, percentages of the number of days when the evaluation was −, +, ++ of the total observation days are shown. a): Significant (p<0.05) relative to the absence of drinking.
Experiment 3.

The sleep apnea suppressing effect of glycine was evaluated. The air flow in the mouth-nose part during sleep and arterial blood oxygen saturation (SpO₂) were measured for each case of administration of glycine tablet (4 tablets) before bedtime and no administration and the effects of glycine on decreased SpO₂ and frequency of apnea occurrence were evaluated. Tracheal sounds during sleep, which is considered to reflect the snoring sound, were simultaneously recorded.

Results of Experiment 3.

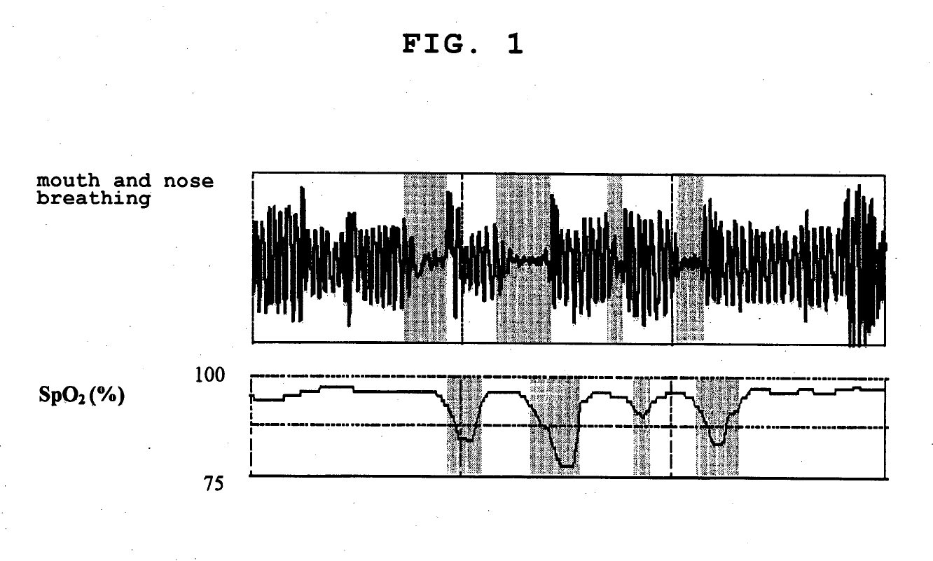

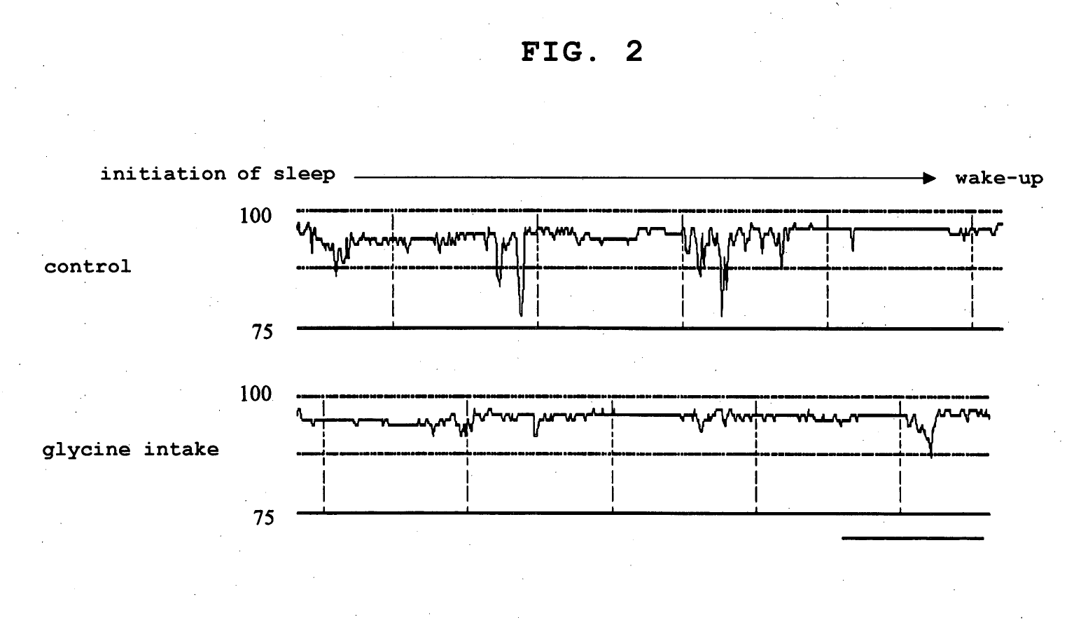

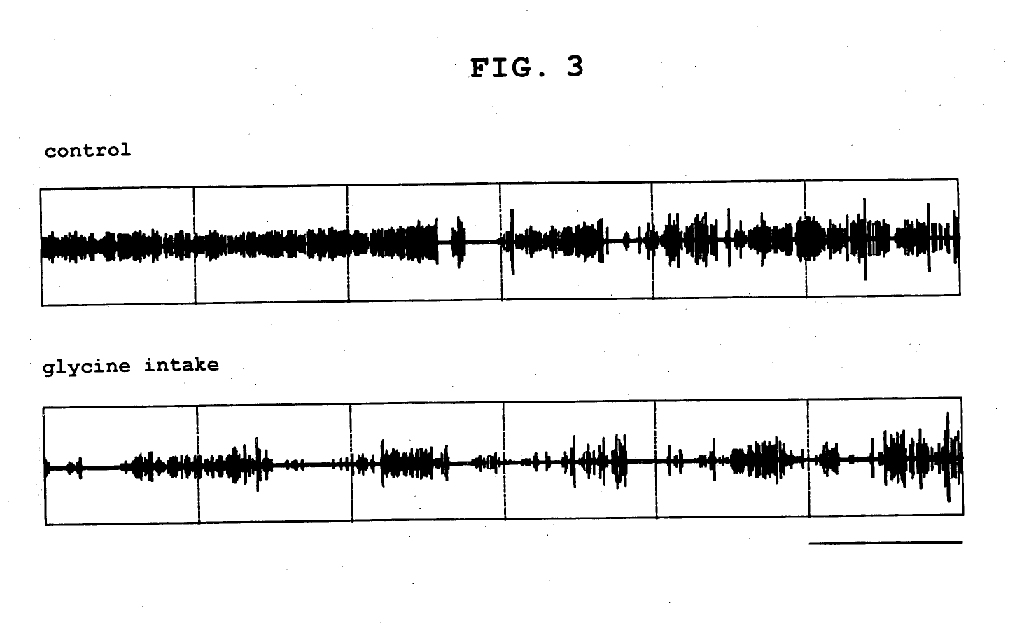

As shown in Table 4 below, administration of glycine tablets resulted in a decrease in the number of apnea per night to 40% as compared to non-administration of glycine. The frequency of apnea per hour also decreased drastically from 4.5 times to 1.9 times by glycine tablet administration. As shown in FIG. 1, a decrease in the arterial blood oxygen saturation (SpO₂) was associated with apnea. Due to the administration of glycine tablets, the frequency of SpO² decrease was suppressed to about 30% as compared to non-administration of glycine (see, Table 4). FIG. 2 shows a profile of arterial blood oxygen saturation during sleep. Due to the administration of glycine tablets, SpO₂ decrease was remarkably suppressed, which in turn increased the lowest value of arterial blood oxygen saturation as shown in Table 4. For a comparison of the occurrence of snoring between glycine administration and non-administration, the tracheal sounds were simultaneously monitored, and a typical example is shown in FIG. 3. The tracheal sounds at the time of glycine tablet administration tended to be suppressed as compared to the non-administration control group. The palliation of SpO₂ decrease due to glycine tablet administration is considered to be the result of the suppression of sleep apnea by glycine, and this effect is considered to be also related to the suppression of occurrence of snoring by glycine.

TABLE 4

| glycine | apnea | | SpO₂ decrease | | |
|---|---|---|---|---|---|
| administration | (times/ | (times/ | (times/ | (times/ | lowest |
| (4 tablets) | day) | hr) | day) | hr) | value |
| − | 27 ± 10 | 4.5 ± 1.3 | 46 ± 3 | 7.8 ± 0.2 | 80 ± 4% |
| + | 11 ± 4 | 1.9 ± 0.9 | 14 ± 5 | 2.5 ± 1.2 | 88 ± 1% |
| | | | a) | a) | |

Using a sleep tester LT-200, mouth-nose breathing and arterial blood oxygen saturation (SpO₂) during sleep were measured, and the number of apnea and SpO₂ decrease per day and per one hour are shown for each case of tablet non-administration and glycine tablet administration (4 tablets). In addition, the observed lowest value of SpO₂ during sleep is shown for each case of tablet nonadministration and glycine tablet administration. a): significant against no administration of glycine tablet (p<0.05, student's t-test).

Snoring prevents sleep of surrounding people as well as snorer and could be the cause of daytime fatigue, lack of concentration and, sometimes, a serious accident. A decrease in the arterial blood oxygen saturation due to sleep apnea also stimulates wakefulness, which in turn causes a pathologic sleep disorders. Moreover, it is considered that the decreased arterial blood oxygen saturation is one cause of circulatory diseases and the like. In the present Examples, it has been clarified that glycine suppresses the occurrence of snoring and alleviates sleep apnea, and further that glycine markedly suppresses a decrease in the arterial blood oxygen saturation during sleep. These effects of glycine provide high quality sleep, which is effective for the prevention of daytime fatigue, enhancement of vitality, reduction of the risk of serious accidents caused by lack of concentration or dozing, and further for the prophylaxis of various diseases caused by sleep apnea. In addition, since the action of glycine is considered to be suppression of airway constriction during sleep, glycine can be effectively used for suppressing a decrease in the arterial blood oxygen saturation during sleep, which is due to a disease such as asthma and the like.

### Example 2

For confirmation of the effect of glycine on respiratory disorders during sleep, a double blind crossover test was performed with 10 male adults having sleep apnea syndrome as evidenced by more than 5 of the following AHI (apnea hypopnea index per 1 hour). Glycine (3 g) was administered 1 hour before sleep and, as the placebo, maltose adjusted to an indistinguishable taste was used.

Evaluation of Hypopnea or Apnea.

Sleep polygraph data such as electroencephalogram, electromyogram, arterial blood oxygen saturation, and the like during sleep were measured using the Alice system manufactured by Respironics Co., Ltd. (USA). The air flow of the mouth-nose part was measured, and hypopnea was defined to mean a state where a decrease to not more than 50% requires not less than 10 second to recover to not less than 60%. Furthermore, a decrease in the arterial blood oxygen saturation by not less than 3% associated with hypopnea was taken as apnea. The total of the incidents of apnea and hypopnea per 1 hour was taken as an apnea hypopnea index (AHI) and used as a severity index of respiratory disorder during sleep.

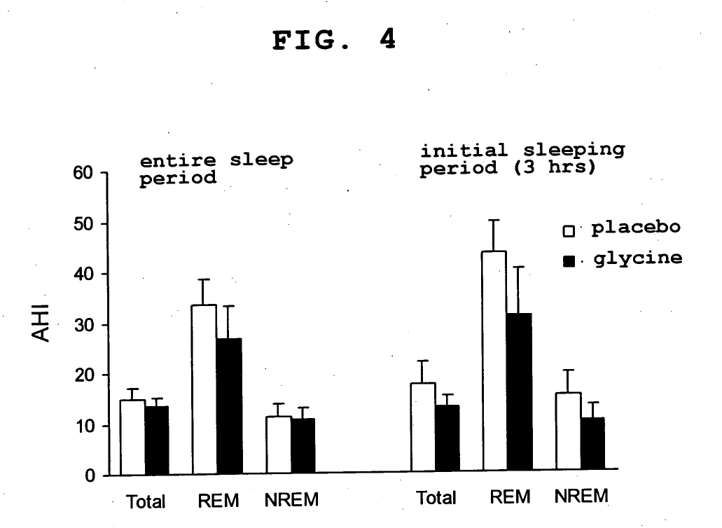

The breathing state during sleep after taking glycine or placebo was measured by the above-mentioned sleeping polygraph examination. As a result, an average value of AHI after taking glycine was lower for any of the entire period (Total) of the rem sleep stage and the nonrem sleep stage, the rem sleep stage (REM) and the nonrem sleep stage (NREM) than the average value of AHI after taking the placebo. Namely, as shown in FIG. 4, left graph, the average value of AHI after taking glycine versus the average value of AHI after taking the placebo was 13.4 versus 15.0 for the entire period (Total) of the rem sleep stage and the nonrem sleep stage, 26.8 versus 33.5 for the rem sleep stage (REM), and 10.8 versus 11.3 for the nonrem sleep stage (NREM). From the foregoing results, it is clear that the glycine administration improved the respiratory disorder during sleep.

As shown in FIG. 4 right graph, moreover, hypopnea or apnea was particularly frequently observed in an early stage of sleep (3 hours) after taking placebo, but the respiratory disorder in the early stage of sleep was improved by glycine administration.

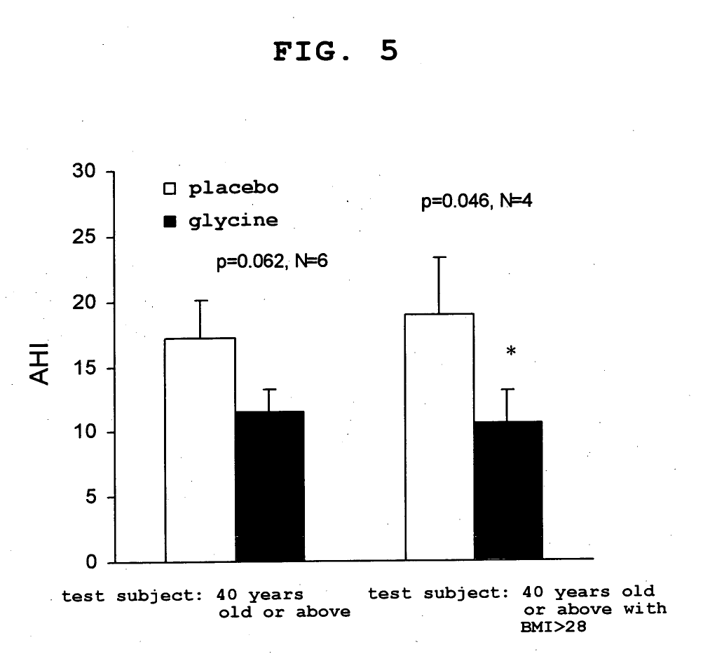

Particularly, in the test recipients of an age of 40 years old or over (6 cases), a remarkable effect of improving respiratory disorders by glycine administration was observed over the entire sleep stage. Namely, as shown in FIG. 5 left graph, an average value of AHI after taking glycine versus average value of AHI after taking placebo was 11.5 versus 17.2 (p=0.062). Specifically, in the test recipients of an age of 40 years old or over, who show an obesity index (BMI) exceeding 28 (4 cases), a significant effect of improving respiratory disorders by glycine administration was observed. Namely, as shown in FIG. 5 right graph, an average value of AHI after taking glycine versus average value of AHI after taking placebo was 10.6 versus 18.9 (p=0.046). The test was a t-test (paired t-test) and p values of less than 0.05 were taken to be significant.

The foregoing results demonstrate that glycine is effective for improving respiratory disorders over the entire sleep stage, and particularly highly effective in the early stage of sleep. It has been also demonstrated that glycine has an effect of remarkably improving respiratory disorders during sleep in the test recipients of an age of 40 years old or over, particularly the test recipients of an age of 40 years old or over, who show a high degree of obesity (BMI>28).

In general, sleep is divided into a nonrem sleep and a rem sleep, and it is said that the occurrence of hypopnea/apnea may vary depending on the state of sleep; in other words, the frequency may change between rem sleep and nonrem sleep. Rem sleep is characterized by rapid eye movements during sleep. Rem sleep and nonrem sleep are each known to play an important role for maintenance of health.

As is clear from the results of Example 2 (FIG. 4), improvements in the respiratory disorders by glycine administration were observed in both periods of rem sleep stage and nonrem sleep stage in both the entire sleep stage and an early stage of sleep (3 hours).

### INDUSTRIAL APPLICABILITY

The agent for the prophylaxis or treatment of the present invention effectively prevents snoring and remarkably suppresses sleep apnea. Therefore, it is effective for the prophylaxis and/or treatment of what is called a sleep apnea syndrome, and moreover, is also effective for the prophylaxis and/or treatment of respiratory disorders caused by other diseases such as asthma and the like.

As a result of improvement in hypopnea/apnea by the agent for the prophylaxis or treatment of the present invention, high quality sleep can be obtained, which in turn prevents daytime somnolence, the scattering of attention, decline of memory, reduced efficiency of work, accidents, frustration, short temper, morning headaches, dysthymia, hyposexuality, hallucination, deafness, and the like due to sleep disorder, as well as abnormal behavior during night due to sleep disorder, and further, social maladjustment due to various symptoms caused by these sleep disorders and anxiety or melancholia.

In other words, as a consequence, it prevents daytime fatigue, enhances vitality, reduces the risk of serious accidents caused by lack of concentration or by dozing, and further prevents various diseases such as cerebrovascular disorder, hypertension, ischemic heart disease, arrhythmia, sudden death, etc. caused by sleep apnea.

Since glycine etc. and glycine receptor agonists, which are the active ingredients of the agent for the prophylaxis or treatment of the present invention, have established safety, the agent for the prophylaxis or treatment of the present invention is highly safe and can be used not only for pharmaceuticals but also in foods and diets.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An agent for the prophylaxis or treatment of snoring or a respiratory disorder during sleep, which comprises either: (a) a compound having an agonist action on a glycine receptor as an active ingredient; or (b) a compound capable of being present as glycine in living organisms as an active ingredient.

2. The agent of claim 1, which comprises a compound capable of being present as glycine in living organisms as an active ingredient.

3. The agent of claim 2, wherein said compound capable of being present as glycine in living organisms is at least one compound selected from the group consisting of glycine, serine, a peptide containing glycine and/or serine as component amino acid(s), a protein containing glycine and/or serine as component amino acid(s), and mixtures thereof.

4. The agent of claim 1, wherein said respiratory disorder during sleep is hypopnea or apnea.

5. The agent of claim 4, wherein said hypopnea or apnea during sleep is obstructive hypopnea, obstructive sleep apnea, central nervous system hypopnea, or central nervous system apnea.

6. The agent of claim 1, which is an agent for the prophylaxis or treatment of sleep apnea syndrome.

7. The agent of claim 1, wherein said respiratory disorder during sleep is a respiratory disorder caused by asthma.

8. The agent of claim 2, which is used for human or animal, wherein the intake or dose is 0.06 to 2500 mg/kg/day based on the free form of glycine, or 0.1 to 4000 mg/kg/day based on the free form of serine.

9. A food for the prophylaxis or palliation of snoring or a respiratory disorder during sleep, which comprises either: (a) a compound having an agonist action on a glycine receptor; or (b) a compound capable of being present as glycine in living organisms.

10. The food of claim 9, which comprises a compound capable of being present as glycine in living organisms.

11. The food of claim 9, which is in the form of a beverage.

12. The food of claim 9, which is a supplement.

13. The food of claim 9, which is a food with health claims.

14. A diet for the prophylaxis or palliation of snoring or a respiratory disorder during sleep, which comprises either: (a) a compound having an agonist action on a glycine receptor; or (b) a compound capable of being present as glycine in living organisms.

15. The diet of claim 14, which comprises a compound capable of being present as glycine in living organisms.

16. A package comprising the agent of claim 1 together with a written matter describing items relating to the use of the agent for the prophylaxis or treatment of snoring or a respiratory disorder during sleep.

17. A package comprising the agent of claim 2 together with a written matter describing items relating to the use of the agent for the prophylaxis or treatment of snoring or a respiratory disorder during sleep.

18. A method for the prophylaxis or treatment of snoring or a respiratory disorder during sleep, which comprises administering to a subject in need thereof an effective amount of either: (a) a compound having an agonist action on a glycine receptor; or (b) a compound capable of being present as glycine in living organisms.

19. The method of claim 18, which comprises administering a compound capable of being present as glycine in living organisms.

20. The method of claim 19, wherein said compound capable of being present as glycine in living organisms is at least one compound selected from the group consisting of glycine, serine, a peptide containing glycine and/or serine as component amino acid(s), a protein containing glycine and/or serine as component amino acid(s), and mixtures thereof.

21. The method of claim 18, wherein said respiratory disorder during sleep is hypopnea or apnea.

22. The method of claim 21, wherein said hypopnea or apnea during sleep is obstructive hypopnea, obstructive apnea, central nervous system hypopnea, or central nervous system apnea.

23. The method of claim 18, which is a method for the prophylaxis or treatment of sleep apnea syndrome.

24. The method of claim 18, wherein said respiratory disorder during sleep is a respiratory disorder caused by asthma.

25. The method of claim 19, which comprises administering, to a human or animal, said compound capable of being present as glycine in living organisms in ana amount of 0.06 to 2500 mg/kg/day based on the free form of glycine or 0.1 to 4000 mg/kg/day based on the free form of serine.

26. A method for the prophylaxis or palliation of snoring or a respiratory disorder during sleep, which comprises feeding to a subject in need thereof an effective amount of either: (a) a food containing a compound having an agonist action on a glycine receptor; or (b) a food containing a compound capable of being present as glycine in living organisms.

27. The method of claim 26, which comprises feeding to said subject a food containing a compound capable of being present as glycine in living organisms.

28. The method of claim 26, wherein said food is in the form of a beverage.

29. The method of claim 26, wherein said food is a supplement.

30. The method of claim 26, wherein said food is a food with health claims.

31. A method for the -prophylaxis or palliation of snoring or a respiratory disorder during sleep, which comprises feeding to a subject in need thereof an effective amount of either: (a) a diet containing a compound having an agonist action on a glycine receptor; or (b) a diet containing a compound capable of being present as glycine in living organisms.

32. The method of claim 31, which comprises feeding to said subject a diet containing a compound capable of being present as glycine in living organisms.
